(19) United States
(12) Patent Application Publication
(10) Pub. No.: US 2007/0233032 A1
(43) Pub. Date: Oct. 4, 2007

(54) Disposable diaper changing kit

(76) Inventor: Beth L. Rau, Westminster, MA (US)

Correspondence Address: LITMAN LAW OFFICES, LTD., P.O. BOX 15035, CRYSTAL CITY STATION, ARLINGTON, VA, 22215, US

(21) Appl. No.: 11/727,801

(22) Filed: Mar. 28, 2007

Related U.S. Application Data
(60) Provisional application No. 60/787,223, filed on Mar. 30, 2006.

(51) Int. Cl. A61F 13/15

(52) U.S. Cl. 604/385.02; 604/385.06; 206/438

(57) ABSTRACT

The disposable diaper changing kit includes a disposable package having a front wall and a rear wall of double wall construction including an inner rear wall and an outer rear wall. The front wall and inner rear wall are joined to one another to define a first pocket, with their top edges being releasably joined to one another by a releasable fastener, such as a bead and groove-type fastener, for example. The inner rear wall and outer rear wall are joined around their peripheral edges to define a second pocket. A disposable diaper and a disposable diaper changing mat are received within the first pocket. At least one disposable baby wipe is packaged within the second pocket. A slot is formed by perforations through the outer rear wall for providing access to, and dispensing of, the disposable wipe. The slot may be releasably sealed by an adhesive strip.

### CROSS-REFERENCE TO RELATED APPLICATION

This application claims the benefit of U.S. Provisional Patent Application Ser. No. 60/787,223, filed Mar. 30, 2006.

### BACKGROUND OF THE INVENTION

1. Field of the Invention

The present invention relates to disposable diapers, and particularly to a disposable diaper changing kit including a resealable package containing a disposable diaper, a diaper changing pad, and disposable wipes.

2. Description of the Related Art

Disposable diapers, disposable diaper changing pads and disposable sanitary wipes for infants are often sold in separate portable packages to be transported with an infant. Typically, each item comes in its own single-use package, and the packages are often carried in a large diaper changing bag, or the like. It would be desirable to provide a single package containing all of the items necessary for the diaper changing of the infant, rather than a plurality of separate packages that must be carried in a larger bag.

Further, such packages typically provide an envelope, which is torn open, or otherwise opened, so that the particular item housed within can be removed. Since these packages are typically intended to be single-use packages, the envelope-type housings tend not to be resealable. Consequently, such packages cannot be used to seal up a used, disposable diaper for disposal. Thus, a disposable diaper changing kit solving the aforementioned problems is desired.

### SUMMARY OF THE INVENTION

The disposable diaper changing kit includes a disposable package having a front wall and a rear wall. The rear wall has a double wall structure including an inner rear wall and an outer rear wall. The front wall and inner rear wall are permanently joined to one another along their bottom and side edges to define a first interior region or pocket therebetween. The top edges of the front wall and interior rear wall are releasably joined to one another by a releasable fastener, such as a Ziploc® seal (Ziploc is a registered trademark of S.C. Johnson Home Storage, Inc. of Racine, Wis.) with an elongated mating bead and groove across the opening.

The inner rear wall and outer rear wall are joined together along their peripheral edges to define a second interior region or pocket therebetween. At least one disposable diaper and at least one disposable diaper changing mat are received within the first pocket. One or more disposable baby wipes are packaged within the second interior region. A slot is formed through the outer rear wall for providing access to and dispensing the disposable wipes. The slot may be closed by a perforated closure, which is covered by an adhesive strip to seal in the wipes to keep them moist with baby oil, baby wash, or other baby wipe cleaning formula.

In use the soiled diaper and used changing pad may be placed in the first pocket and sealed with the Ziploc®-type closure and the entire package may be disposed of.

These and other features of the present invention will become readily apparent upon further review of the following specification and drawings.

### BRIEF DESCRIPTION OF THE DRAWINGS

FIG. 1 is a front perspective view of a disposable diaper changing kit according to the present invention.

FIG. 2 is a rear view of a package of the disposable diaper changing kit according to the present invention, partially broken away to show the rear pocket.

FIG. 3 is a rear view of the package of the disposable diaper changing kit according to the present invention, shown dispensing a disposable baby wipe through a perforated opening.

Similar reference characters denote corresponding features consistently throughout the attached drawings.

### DETAILED DESCRIPTION OF THE PREFERRED EMBODIMENTS

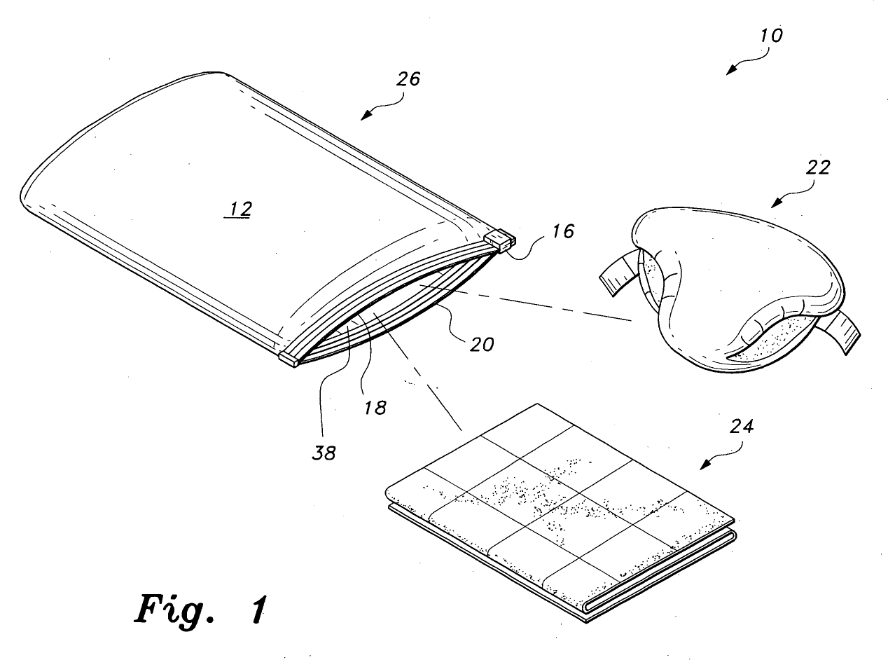

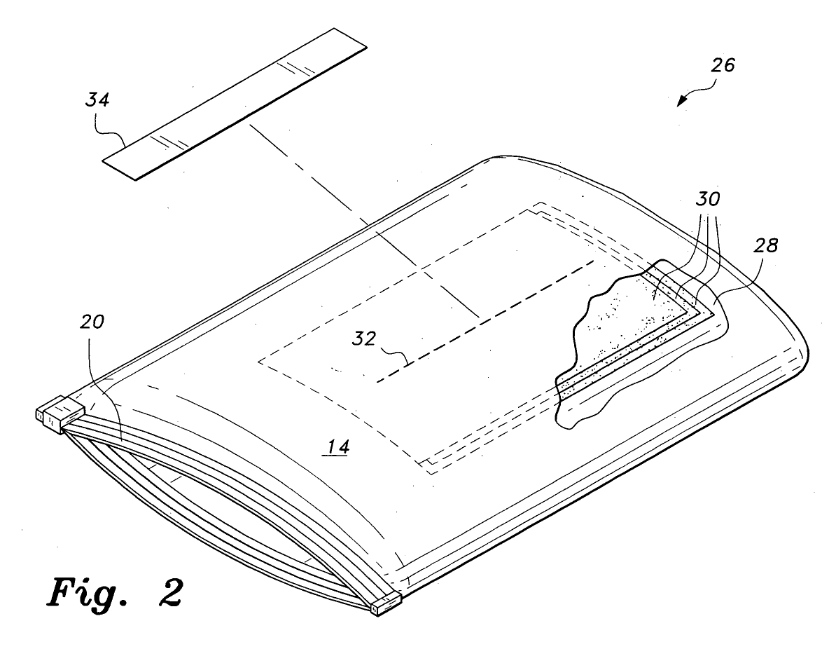

As illustrated in FIGS. 1 and 2, the disposable diaper changing kit 10 includes a disposable package 26 for storing and dispensing at least one disposable diaper 22, at least one disposable diaper changing pad or mat 24, and at least one disposable sanitary wipe 30. Kit 10 is portable and includes materials commonly utilized in the changing of an infant's diaper. Following diaper changing, the entire kit 10 may be disposed of, although package 26 may be resealed for subsequent usage.

Package 26 includes a front wall 12 and a rear wall having a double wall construction, including an inner rear wall 28 and an outer rear wall 14 (shown best in FIG. 2). Preferably, front wall 12, inner rear wall 28 and outer rear wall 14 have a substantially rectangular contour. However, each wall may have any desired suitable shape or size, dependent upon the particular needs and desires of the user. Front wall 12, inner rear wall 28, and outer rear wall 14 may be formed from flexible plastic or any other suitable lightweight, disposable, easily transportable, fluid-tight material. The bottom and side edges of the front wall 12 and inner rear wall 28 are joined together through heat sealing or any other suitable method to define a first open interior region or pocket 38 therebetween.

The top edge 18 of front wall 12 and the top edge 20 of inner rear wall 28 are releasably sealed by releasable closure 16. Releasable closure 16 may be any suitable releasable closure which forms a fluid-tight seal between edges 18 and 20, dependent upon the particular needs and desires of the user. In the preferred embodiment, releasable closure 16 is a bead and groove fastener, as are commonly found on resealable plastic sandwich bags. One such fastener is sold under the mark Ziploc®, and is manufactured by S.C. Johnson Home Storage, Inc. Alternatively, a secondary seal may be provided for selectively preventing access to the bead and groove fastener until the user opens the package for the first time. Such a seal may, for example, be a perforated closure, joining edges 18 and 20 above the bead and groove fastener, which would be torn open by the user to access the bead and groove fastener upon initial opening of the package.

At least one disposable diaper 22 and at least one disposable diaper changing pad or mat 24 are removably stored within first pocket 38. If multiple diapers 22 and/or mats 24 are provided, or if the package is accidentally opened, the releasable closure 16 allows package 26 to be resealed for subsequent usage. Further, the soiled diaper removed from the infant and the used changing pad can be placed within pocket 38 and the fluid-tight closure 16 may be sealed so that the entire package 26 can be transported to a waste disposal site in a sanitary manner.

As shown in FIG. 2, outer rear wall 14 and inner rear wall 28 are joined at their peripheral edges to define a second interior region or pocket therebetween. At least one baby wipe 30 is packaged or stored within the second pocket, sandwiched between the outer rear wall 14 and the inner rear wall 28. Because of this construction, it is unnecessary to individually wrap each disposable baby wipe. Outer rear wall 14 is formed with a plurality of perforations 32 defined therein. Perforations 32 are preferably arranged in a line segment extending substantially laterally across a central portion of outer rear wall 14, as shown in FIG. 2.

Baby wipes, particularly for use with infants, are often treated with lotions, such as baby oil, baby wash, cleaning fluids, aloe, and other liquids. In order to prevent evaporation of these liquids through perforations 32, a removable seal, such as adhesive strip 34, is provided for covering perforations 32. Adhesive strip 34 may be adhesive tape or the like.

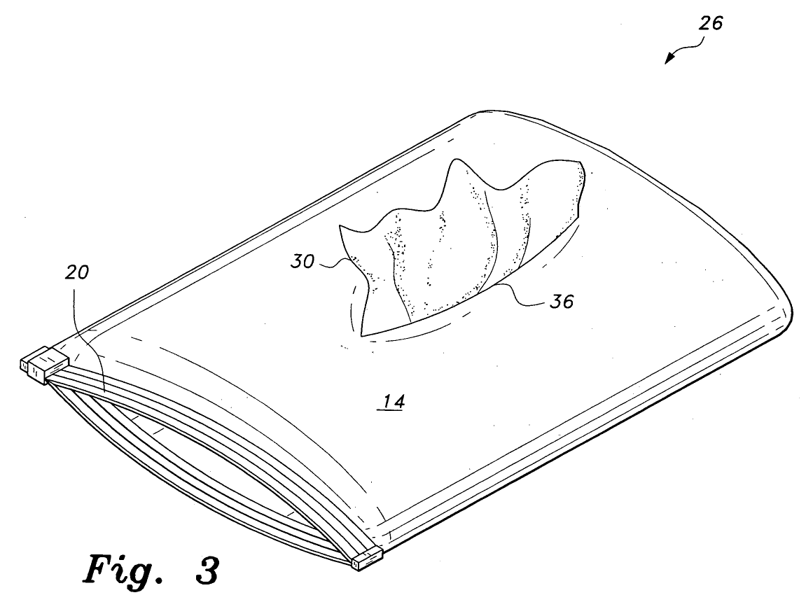

In use, the user tears the perforated seal 32 to form a slot 36, as shown in FIG. 3. If more than one baby wipe 30 is provided, the baby wipes may be folded within the interior region in such a way that they can be continuously dispensed, in a manner commonly associated with facial tissue dispensers. Further, if a portion of the baby wipes 30 remains within the interior region following the diaper changing procedure, the slot 36 may be resealed by application of adhesive strip 34 to cover and reseal the slot for subsequent usage, if desired.

Kit 10 provides the necessary materials for infant diaper changing, initially housed in an easily transportable and disposable package. The package 26 and materials may be used a single time and disposed of, or the package 26 may be resealed and transported for subsequent usage. Further, package 26 may be used to transport a dirty diaper, a used diaper changing pad or mat, and/or used baby wipes to a waste storage site.

It is to be understood that the present invention is not limited to the embodiments described above, but encompasses any and all embodiments within the scope of the following claims.

I claim:

1. A disposable diaper changing kit, comprising:
a package having a front wall and a rear wall, the rear wall being a double wall having an inner rear wall and an outer rear wall, the front wall and the rear wall each having a lower edge and at least one side edge joined together to form a first pocket, the inner and outer rear walls having peripheral edges joined together to define a second pocket, the outer rear wall having a row of perforations formed therethrough defining a slot, the front and rear wall each further having a top edge, the top edges defining an opening to the first pocket;
means for releasably joining the top edges of the front and rear walls;
a disposable diaper removably received within the first pocket;
a diaper changing pad removably received within the first pocket; and,
at least one disposable baby wipe removably received within the second pocket.

2. The disposable diaper changing kit as recited in claim 1, wherein said means for releasably joining the top edges of said front wall and said rear wall comprise an elongated bead and groove fastener disposed along the top edges of said front wall and said rear wall.

3. The disposable diaper changing kit as recited in claim 1, further comprising an adhesive strip releasably covering said perforations.

4. The disposable diaper changing kit as recited in claim 1, wherein said package has a substantially rectangular contour.

5. The disposable diaper changing kit as recited in claim 4, wherein said second pocket has a substantially rectangular contour.

6. The disposable diaper changing kit as recited in claim 1, wherein said package is formed from disposable materials.

7. The disposable diaper changing kit as recited in claim 1, wherein said at least one disposable baby wipe comprises a plurality of disposable baby wipes.
